(19) United States
(12) Patent Application Publication
(10) Pub. No.: US 2026/0335878 A1
(43) Pub. Date: Oct. 29, 2026

(54) FLOW PATH SYSTEM FOR GENE SEQUENCING, CONTROL METHOD THEREOF AND GENE SEQUENCER

(71) Applicant: WUHAN MGI TECH CO., LTD., Wuhan (CN)

(72) Inventors: Xingye GUI, Wuhan (CN); Jian YE, Wuhan (CN); Jing YE, Wuhan (CN); Qian ZHANG, Wuhan (CN); Tingting LU, Wuhan (CN); Jiwei HE, Wuhan (CN); Fen LIU, Wuhan (CN); Heming JIANG, Wuhan (CN)

(21) Appl. No.: 19/485,285

(22) PCT Filed: Jul. 5, 2023
(86) PCT No.: PCT/CN2023/105922

§ 371 Date: Nov. 18, 2025

(51) Int. Cl. B01L 3/00; F16K 11/08

(52) U.S. Cl. CPC B01L 3/502; B01L 3/527; B01L 13/02; B01L 2200/026; B01L 2200/0631; B01L 2200/10; B01L 2200/16; B01L 2300/0822; B01L 2300/0861; B01L 2400/049; B01L 2400/0622; B01L 2400/0644; F16K 11/08

(57) ABSTRACT

The present disclosure provides a flow path system for gene sequencing, which comprises: a sequencing slide including a plurality of sample channels independent from each other; and at least one of the following: a biological sample loading flow path configured to load different biological samples in at least two sample channels respectively, and/or selectively loading one biological sample into some of the plurality of sample channels; a sequencing reagent loading flow path configured to be capable of selectively loading a plurality of different sequencing reagents into some of the sample channels respectively; and a rinsing flow path configured to rinse a component in the flow path system using a plurality of rinsing agents respectively. Accordingly, it is realized that different samples are loaded in at least two sample channels respectively, and each flow path member in the flow path system is rinsed using a plurality of different rinsing agents respectively.

### CROSS-REFERENCE TO RELATED APPLICATIONS

The present application is a U.S. National Stage Application under 35 U.S.C. § 371 of International Patent Application No. PCT/CN2023/105922, filed on Jul. 5, 2023, the disclosure of which is incorporated by reference herein in its entirety.

### TECHNICAL FIELD

The present disclosure relates to the technical field of gene sequencer, in particular to a flow path system for gene sequencing, an operation method thereof and a gene sequencer.

### BACKGROUND

The test slide in the gene sequencer is divided into at least two sample channels, and each independent channel can theoretically load different biological samples. However, due to the complexity of the fluid system, the existing device does not possess the function of loading different biological samples in each sample channel respectively, which is required to be completed by special loading tools. Moreover, it is impossible to automatically use a plurality of different rinsing agents to rinse each flow path member, which is inefficient in rinsing and time-consuming and laborious.

### SUMMARY

The present disclosure provides a flow path system for gene sequencing, which comprises: a sequencing slide including a plurality of sample channels independent from each other; and at least one of the following: a biological sample loading flow path for loading different biological samples in at least two sample channels respectively, and/or selectively loading one biological sample into some of a plurality of sample channels; a sequencing reagent loading flow path configured to selectively load a plurality of different sequencing reagents into some of the sample channels respectively; and a rinsing flow path configured to rinse a component in the flow path system using a plurality of rinsing agents respectively.

In some embodiments, the flow path system further comprises at least one of the following: a biological sample area provided with a plurality of sample storage bins for storing a plurality of different biological samples; a sequencing reagent area where a plurality of different sequencing reagents are stored; a rinsing agent area which stores a plurality of different rinsing agents for rinsing a flow path member of the flow path system; and a waste liquid area in communication with the biological sample loading flow path, the sequencing reagent loading flow path and the rinsing flow path so as to receive waste liquid.

In some embodiments, the flow path system comprises: a suction device including a plurality of suction members operating independently of each other for sucking a biological sample; a first directional control valve assembly includes a plurality of first directional control valves operating independently of each other; and three ports of each first directional control valve in communication with a corresponding biological sample storage bin, a corresponding suction member and a corresponding sample channel respectively, so as to realize the selective communication of the suction member between the biological sample storage bin and the sample channel.

In some embodiments, a first port of each first directional control valve communicates with a corresponding biological sample storage bin, a second port of each first directional control valve communicates with an I port of a corresponding suction member, and a third port of each first directional control valve communicates with an inlet of a corresponding sample channel. In the case where the first directional control valve is powered on, the first port communicates with the second port, and the second port is disconnected from the third port. In the case where the first directional control valve is powered off, the second port communicates with the third port, and the first port is disconnected from the second port.

In some embodiments, the flow path system further comprises a rotary valve and a fifth directional control valve, wherein a first port of the fifth directional control valve communicates with the rotary valve, a second port of the fifth directional control valve communicates with a corresponding biological sample storage bin, and a third port of the fifth directional control valve communicates with a first port of one of a plurality of first directional control valves; the rotary valve communicates with the outlet ends of a plurality of sample channels and the waste liquid area; in the case where the fifth directional control valve is powered on, the first port communicates with the second port, and the second port is disconnected from the third port; in the case where the fifth directional control valve is powered off, the first port is disconnected from the second port, and the second port communicates with the third port.

In some embodiments, each outlet of the plurality of suction members remains in communication with the waste liquid area, so that when the plurality of suction members are switched to the outlet, the waste liquid can be discharged into the waste liquid area.

The present disclosure also provides a gene sequencer, which comprises the above-described flow path system.

The present disclosure also provides a method for controlling a flow path system for gene sequencing, wherein the flow path system comprises a biological sample area, a first directional control valve assembly, a sequencing slide, a suction device and a waste liquid area, wherein the biological sample area is provided with a plurality of sample storage bins for accommodating a plurality of biological samples respectively, the first directional control valve assembly includes a plurality of first directional control valves, the sequencing slide is provided with a plurality of sample channels, the suction device includes a plurality of suction members, the first ports of the plurality of first directional control valves are in communication with a plurality of sample storage bins through a plurality of first pipelines respectively, the second ports of the plurality of first directional control valves are in communication with I ports of the plurality of suction members through a plurality of second pipelines respectively, and the third ports of the plurality of first directional control valves are in communication with each inlet end of the plurality of sample channels through a plurality of third pipelines respectively, and a rotary valve is provided between the outlet ends of the plurality of sample channels and the waste liquid area for controlling the on-off between the outlet ends of the plurality of sample channels and the waste liquid area; the method comprises: powering on the plurality of first directional control valves so that the first ports of the plurality of first directional control valves are in communication with the second ports respectively; causing the inlets of a plurality of suction members to generate negative pressure to suck a plurality of samples from a plurality of sample storage bins respectively to the I ports of the plurality of suction members through the first pipeline, the plurality of first directional control valves and the second pipeline, and finally temporarily store the plurality of biological samples in the second pipeline.

The present disclosure also provides a method for controlling a flow path system for gene sequencing, wherein the flow path system comprises a biological sample area, a first directional control valve assembly, a sequencing slide, a suction device and a waste liquid area, wherein the biological sample area is provided with a plurality of sample storage bins for accommodating a plurality of biological samples respectively, the first directional control valve assembly includes a plurality of first directional control valves, the sequencing slide is provided with a plurality of sample channels, the suction device includes a plurality of suction members, the first ports of the plurality of first directional control valves are in communication with a plurality of sample storage bins through a plurality of first pipelines respectively, the second ports of the plurality of first directional control valves are in communication with inlets of a plurality of suction members through a plurality of second pipelines respectively, and the third ports of the plurality of first directional control valves are in communication with each inlet end of a plurality of sample channels through a plurality of third pipelines respectively, and a rotary valve is provided between the outlet ends of the plurality of sample channels and the waste liquid area for controlling the on-off between the outlet ends of the plurality of sample channels and the waste liquid area; wherein the first directional control valve assembly of the flow path system includes a second directional control valve, the first port of the second directional control valve communicates with a first sequencing reagent needle in the sequencing reagent area, the second port of the second directional control valve communicates with B ports of a plurality of suction members, and the third port of the second directional control valve communicates with the outlet ends of a plurality of sample channels and the rotary valve through tees respectively, wherein in the case where each of the first and second directional control valves is powered on, the first port communicates with the second port, and the second port is disconnected from the third port, and in the case where each of the first and second directional control valves is powered off, the second port communicates with the third port, and the first port is disconnected from the second port; the method comprises: powering off a plurality of first directional control valves and powering on a second directional control valve; and causing a plurality of suction members to provide negative pressure so that the sequencing reagent enters the plurality of suction members from the sequencing reagent area through the second directional control valve and the B ports of the plurality of suction members for temporary storage.

The present disclosure also provides a method for controlling a flow path system for gene sequencing, wherein the flow path system comprises a biological sample area, a first directional control valve assembly, a sequencing slide, a suction device and a waste liquid area, wherein the biological sample area is provided with a plurality of sample storage bins for accommodating a plurality of biological samples respectively, the first directional control valve assembly includes a plurality of first directional control valves, the sequencing slide is provided with a plurality of sample channels, and the suction device includes a plurality of suction members, the first ports of the plurality of first directional control valves are in communication with a plurality of sample storage bins through a plurality of first pipelines respectively, the second ports of the plurality of first directional control valves are in communication with inlets of a plurality of suction members through a plurality of second pipelines respectively, and the third ports of the plurality of first directional control valves are in communication with each inlet end of a plurality of sample channels through a plurality of third pipelines respectively, and a rotary valve is provided on a pipeline between the outlet ends of the plurality of sample channels and the waste liquid area for controlling the on-off between the outlet ends of the plurality of sample channels and the waste liquid area; wherein the first directional control valve assembly of the flow path system further includes a second directional control valve, the first port of the second directional control valve communicates with a first sequencing reagent needle in the sequencing reagent area, the second port of the second directional control valve communicates with B ports of a plurality of suction members, and the third port of the second directional control valve communicates with the outlet ends of a plurality of sample channels and the rotary valve through tees respectively; a fifth directional control valve is provided on a first pipeline between one of the plurality of first directional control valves and a corresponding sample storage bin, the first port of the fifth directional control valve communicates with the rotary valve, the second port of the fifth directional control valve communicates with the fourth sample storage bin, the third port of the fifth directional control valve communicates with the first port of one of the plurality of first directional control valves, wherein in the case where each of the first, second and a fifth directional control valve is powered on, the first port communicates with the second port, and the second port is disconnected from the third port, and in the case where each of the first, second and a fifth directional control valve is powered off, the second port communicates with the third port, and the first port is disconnected from the second port; the method comprises: powering on a plurality of first directional control valves and a fifth directional control valve and powering off a second directional control valve; causing one of the plurality of suction members to provide negative pressure to suck a biological sample out of a sample storage bin, and sequentially pass through a sample needle, the fifth directional control valve, the rotary valve and the second directional control valve to reach the B ports of the plurality of suction members, so that a fifth pipeline from the rotary valve to the outlet ends of the plurality of sample channels is filled with the biological sample at this time.

The present disclosure provides a method for controlling a flow path system for gene sequencing, wherein the flow path system comprises a biological sample area, a first directional control valve assembly, a sequencing slide, a suction device and a waste liquid area, wherein the biological sample area is provided with a plurality of sample storage bins for accommodating a plurality of biological samples respectively, the first directional control valve assembly includes a plurality of first directional control valves, the sequencing slide is provided with a plurality of sample channels, and the suction device includes a plurality of suction members, the first ports of the plurality of first directional control valves are in communication with a plurality of sample storage bins through a plurality of first pipelines respectively, the second ports of the plurality of first directional control valves are in communication with inlets of a plurality of suction members through a plurality of second pipelines respectively, and the third ports of the plurality of first directional control valves are in communication with each inlet end of a plurality of sample channels through a plurality of third pipelines respectively, and a rotary valve is provided between the outlet ends of the plurality of sample channels and the waste liquid area for controlling the on-off between the outlet ends of the plurality of sample channels and the waste liquid area; wherein the first directional control valve assembly of the flow path system further includes a second directional control valve, the first port of the second directional control valve communicates with a first sequencing reagent needle in the sequencing reagent area, the second port of the second directional control valve communicates with B ports of a plurality of suction members, and the third port of the second directional control valve communicates with the outlet ends of a plurality of sample channels and the rotary valve through tees respectively; the sequencing reagent area is provided with a plurality of second sequencing reagent needles corresponding to a plurality of different sequencing reagents respectively; the rotary valve is provided with a plurality of hole positions communicating with the plurality of second sequencing reagent needles respectively; when one of the hole positions of the rotary valve is switched, the rotary valve communicates with one of the second sequencing reagent needles corresponding to the hole position; in the case where each of the first and second directional control valves is powered on, the first port communicates with the second port, and the second port is disconnected from the third port, and in the case where each of the first and second directional control valves is powered off, the second port communicates with the third port, and the first port is disconnected from the second port; the method comprises at least one of Bypass operation and Byship operation: Bypass operation, which includes the following steps: (1) powering on a plurality of first directional control valves and powering off a second directional control valve, switching the rotary valve to communicate with a required sequencing reagent through a second sequencing reagent needle, switching a plurality of suction members to a port B, providing negative pressure to suck a required sequencing reagent from the sequencing reagent area, and passing through the second sequencing reagent needle, the rotary valve, the fifth pipeline, the second directional control valve and B ports of a plurality of suction members so that the sequencing reagent finally enters the plurality of suction members; (2) causing the sequencing reagent to enter the plurality of suction members, or as long as it is satisfied to pass through the second directional control valve; (3) switching the plurality of suction members to an outlet, and providing positive pressure to convey the sequencing reagent from the plurality of suction members to the waste liquid area through a fourth pipeline; and Bychip operation, which includes the following steps: (1) powering off a plurality of first directional control valves, powering on a second directional control valve, switching a rotary valve to communicate with a required sequencing reagent through a second sequencing reagent needle, switching a plurality of suction members to an inlet, and causing a plurality of suction members to provide negative pressure and allowing a required sequencing reagent to pass through the second sequencing reagent needle, the rotary valve, the fifth channel and the outlet end of the sequencing slide to enter a biological channel, so as to replace a previous old sequencing reagent therein, so that the old sequencing reagent and redundant new sequencing reagents are discharged from the inlet end of the sequencing slide, and pass through the third pipeline, the plurality of suction members, the second pipeline and inlets of the plurality of suction members to enter the plurality of suction members; (2) switching a plurality of suction members to an outlet, and providing positive pressure thrust to push out a sequencing reagent inside the same, and discharge the same into the waste liquid area through a fourth pipeline.

The present disclosure provides a method for controlling a flow path system for gene sequencing, wherein the flow path system comprises a biological sample area, a first directional control valve assembly, a sequencing slide, a suction device, a waste liquid area and a rinsing agent area, wherein the biological sample area is provided with a plurality of sample storage bins for accommodating a plurality of biological samples respectively; the first directional control valve assembly includes a plurality of first directional control valves and a second directional control valve; the sequencing slide is provided with a plurality of sample channels; the suction device includes a plurality of suction members; the first ports of the plurality of first directional control valves are in communication with a plurality of sample storage bins through a plurality of first pipelines respectively, the second ports of the plurality of first directional control valves are in communication with inlets of a plurality of suction members through a plurality of second pipelines respectively, and the third ports of the plurality of first directional control valves are in communication with each inlet end of a plurality of sample channels through a plurality of third pipelines respectively, and a rotary valve is provided between the outlet ends of the plurality of sample channels and the waste liquid area for controlling the on-off between the outlet ends of the plurality of sample channels and the waste liquid area; wherein the first port of the second directional control valve communicates with a first sequencing reagent needle in the sequencing reagent area, the second port of the second directional control valve communicates with B ports of a plurality of suction members, and the third port of the second directional control valve communicates with the outlet ends of a plurality of sample channels and the rotary valve through tees respectively; a fifth directional control valve is provided on a first pipeline between one of a plurality of first directional control valves and a corresponding sample storage bin, a first port of the fifth directional control valve communicates with a rotary valve, a second port of the fifth directional control valve communicates with a sample storage bin, and a third port of the fifth directional control valve communicates with a first port of a directional control valve; the flow path system also comprises a rinsing agent area and a second valve group, wherein the rinsing agent area stores a plurality of different types of rinsing agents, and the second valve group includes a plurality of inlets and a common outlet, wherein the plurality of inlets are in communication with the rinsing agents of different types respectively, and the common outlet is capable of selectively communicating with an outer wall of the second sequencing reagent needle and a pipeline between the second directional control valve and the B ports of a plurality of suction members; the common outlet of the second valve group communicates with the second port of the fourth directional control valve, the third port of the fourth directional control valve communicates with the inlet of the pump, the outlet of the pump communicates with the second port of the third directional control valve, the third port of the third directional control valve communicates with the outer walls of the plurality of second sequencing reagent needles, and the third directional control valve communicates with the first port of the fourth directional control valve, a pipeline between the first port of the third directional control valve and the first port of the fourth directional control valve selectively communicates with a pipeline between the second directional control valve and the B ports of a plurality of suction members through a cut-off valve, wherein in the case where each of the first to fifth directional control valves is powered on, the first port communicates with the second port, and the second port is disconnected from the third port; when each of the first to fifth directional control valves is powered off, the second port communicates with the third port, and the first port is disconnected from the second port; the method comprises at least one of the following steps: step a: turning on one of the two-way valves, so that a corresponding rinsing agent is selected, and if the rinsing agent is air, it may be used for emptying a liquid in a pipeline; step b: powering off the third directional control valve and the fourth directional control valve, so that the pump rotates forward to transport a selected rinsing agent from the rinsing agent area to the outer wall of the second sequencing reagent needle in the sequencing reagent area through the second valve group, the fourth directional control valve, the pump and the third directional control valve so as to rinse the outer wall of the second sequencing reagent needle; step c: powering on the third directional control valve and powering off the fourth directional control valve, and rotating the pump forward so that a required rinsing agent is pumped, and passes through the second valve group, the fourth directional control valve, the pump and the third directional control valve to reach the cut-off valve; step d: turning on the cut-off valve, powering on a plurality of first directional control valves and powering off the second directional control valve, so that a selected rinsing agent sequentially passes through the cut-off valve, the second directional control valve, the fifth pipeline, the rotary valve and the second sequencing reagent needle, and finally enters a sequencing reagent tank in the sequencing reagent area; step e: switching the rotary valve to each hole position sequentially, and repeating step d to complete the rinsing of the inner wall of each second sequencing reagent needle and a communication pipeline thereof; step f: performing step a for multiple times to select different rinsing agents respectively, and performing step e to rinse the inner wall of each second sequencing reagent needle and a communication pipeline thereof for multiple times with different rinsing agents; step g: powering on the third directional control valve and powering off the fourth directional control valve, powering on a plurality of first directional control valves and the second directional control valve so that the pump rotates backward, and an inlet of the second valve group communicates with the waste liquid area, so that waste liquid in the sequencing reagent tank of the sequencing reagent area is pumped to the waste liquid area through an inlet; step h: turning on the cut-off valve, powering on the plurality of first directional control valves and powering off the second directional control valve, so that the rotary valve is switched to a blocked hole position, the B and outlets of a plurality of suction members are simultaneously opened, and the rinsing agent output in step c sequentially passes through the one-into-multiple liquid dispensing block and the B and outlets of a plurality of suction members, and is finally discharged through the waste liquid area, so as to complete the rinsing of related pipelines and valves; step i: powering off the third directional control valve and powering on the fourth directional control valve, and closing the pump, so that a selected rinsing agent passes through the outlet of the second valve group and the fourth directional control valve to reach the cut-off valve; step j: turning on the cut-off valve, and powering off the plurality of first directional control valves and the second directional control valve, so that the rotary valve is switched to a designated blocked hole position, so that the plurality of suction members are switched to the inlets to provide negative pressure to suction the rinsing agent which sequentially passes through the cut-off valve, the second directional control valve, the plurality of biological channels of the test slide, the plurality of first directional control valves and the inlets of the plurality of suction members, and finally enters the interior of the plurality of suction members, so as to complete the rinsing of related pipelines and valves; step k: switching a plurality of suction members to the outlet, and providing positive pressure to discharge the rinsing agent therein into the waste liquid area through a fourth pipeline; step 1: turning on the cut-off valve, powering on the plurality of first directional control valves and powering off the second directional control valve, so that the rotary valve 15 is switched to a designated blocked hole position, and a plurality of suction members are switched to the B ports, so as to provide negative pressure to suction the rinsing agent which sequentially passes through the cut-off valve, the one-into-multiple liquid dispensing block and the B ports of the plurality of suction members, and finally enters the interior of the plurality of suction members for temporary storage; step m: powering on the plurality of first directional control valves and powering off the second directional control valve, so that the plurality of suction members are switched to the inlet to provide positive pressure to push the rinsing agent, so that the rinsing agent temporarily stored in the plurality of suction members in steps j and i is pushed out, and discharged into the plurality of sample storage bins in the sample storage area sequentially through the inlets of the plurality of suction members and the plurality of first directional control valves, and finally through the plurality of sample needles, so as to complete the rinsing of related pipelines and valves; step n: switching the rotary valve to the hole position in communication with the fifth directional control valve, powering on the plurality of first directional control valves and powering off the second directional control valve, and powering on the fifth directional control valve, switching the plurality of suction members to the B port, and providing positive pressure, so that the rinsing agent temporarily stored in the plurality of suction members in the step j or i is pushed out, and discharged into a sample storage bin sequentially through the B ports of the plurality of suction members, the one-into-multiple liquid dispensing block, the second directional control valve, the rotary valve and the fifth directional control valve, and finally through the sample needle, so as to complete the rinsing of related pipelines and valves.

The flow path system for gene sequencing according to the present disclosure may at least achieve at least one of the following technical effects: different biological samples are loaded independently in a plurality of sample channels of the same sequencing slide respectively, so as to better meet the sequencing requirements of different biological samples; a plurality of different sequencing reagents are selectively loaded into the selected sample channels to satisfy the requirements of using different sequencing reagents in sequencing respectively; a plurality of different rinsing agents are used to rinse a flow path member of the flow path system respectively to satisfy different rinsing requirements of each component.

### BRIEF DESCRIPTION OF THE DRAWINGS

FIG. 1 is a first example diagram of a flow path system for gene sequencing according to the present disclosure;

FIG. 2 is a second example diagram of a flow path system for gene sequencing according to the present disclosure;

FIG. 3 is a third exemplary diagram of a flow path system for gene sequencing according to the present disclosure;

FIG. 4 is a fourth example diagram of a flow path system for gene sequencing according to the present disclosure;

FIG. 5 is a fifth example diagram of a flow path system for gene sequencing according to the present disclosure;

FIG. 6 is a sixth example diagram of a flow path system for gene sequencing according to the present disclosure;

FIG. 7 is a seventh exemplary diagram of a flow path system for gene sequencing according to the present disclosure;

FIG. 8 is an eighth exemplary diagram of a flow path system for gene sequencing according to the present disclosure;

FIG. 9 is a ninth example diagram of a flow path system for gene sequencing according to the present disclosure;

FIG. 10 is a tenth exemplary diagram of a flow path system for gene sequencing according to the present disclosure;

FIG. 11 is an eleventh exemplary diagram of a flow path system for gene sequencing according to the present disclosure;

FIG. 12 is a twelfth example diagram of a flow path system for gene sequencing according to the present disclosure;

FIG. 13 is a thirteenth exemplary diagram of a flow path system for gene sequencing according to the present disclosure;

FIG. 14 is a fourteenth exemplary diagram of a flow path system for gene sequencing according to the present disclosure;

FIG. 15 is a fifteenth exemplary diagram of a flow path system for gene sequencing according to the present disclosure; and

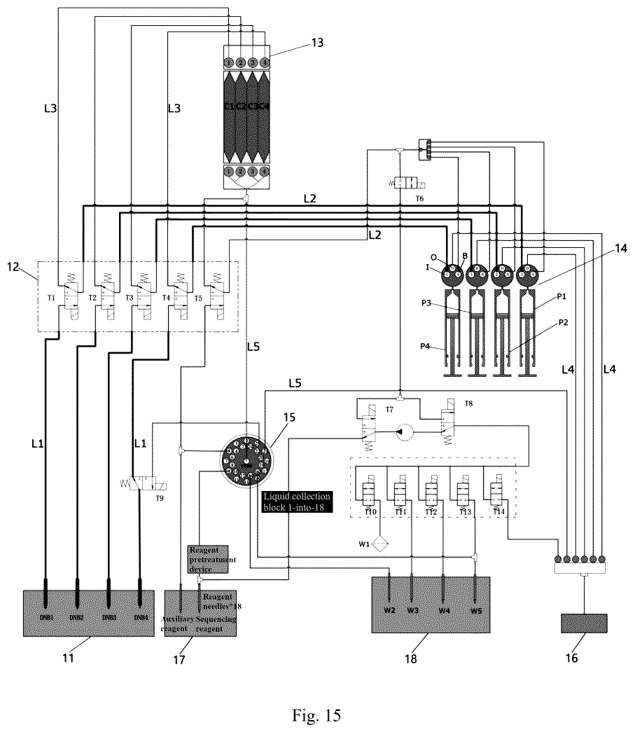

FIG. 16 is a sixteenth exemplary diagram of a flow path system for gene sequencing according to the present disclosure.

### DETAILED DESCRIPTION

In the following, the present disclosure will be further described in detail in conjunction with specific embodiments, and the examples are provided only to elaborate the present disclosure, rather than limiting the scope of the present disclosure.

### Loading Flow Path

The flow path system 10 for gene sequencing according to the present disclosure comprises a biological sample area 11, a first directional control valve assembly 12, a sequencing slide 13 and a suction device 14, wherein the biological sample area 11 is provided with a plurality of sample storage bins for accommodating different biological samples respectively, and the sequencing slide 13 is provided with a plurality of sample channels independent from each other, and the first directional control valve assembly 12 is in communication with the biological sample area 11, the sequencing slide 13 and the suction device 14 respectively. The suction device 14 may be switchably communicated between the biological sample area 11 and the sequencing slide 13 through the first directional control valve assembly 12, so that different biological samples may be sucked and conveyed to a plurality of sample channels of the sequencing slide 13 respectively. The biological sample area 11 may be divided into several sample storage bins independent from each other as required, and the partition contour line is not shown in the accompanying drawings.

In the following, the present disclosure will be described by taking four components as an example. It may be understood that, the above-described embodiments of the present disclosure may also select more or less components, including at least a sample storage bin, a directional control valve, a suction member of a suction device, a sample channel and the like, and may also select some of more or less components to selectively load biological samples into some sample channels. Here, “a plurality of” means at least two, and “first” and “second” are only for distinguishing literally, without any limitation, and “communication” means that they are communicated in the flow path.

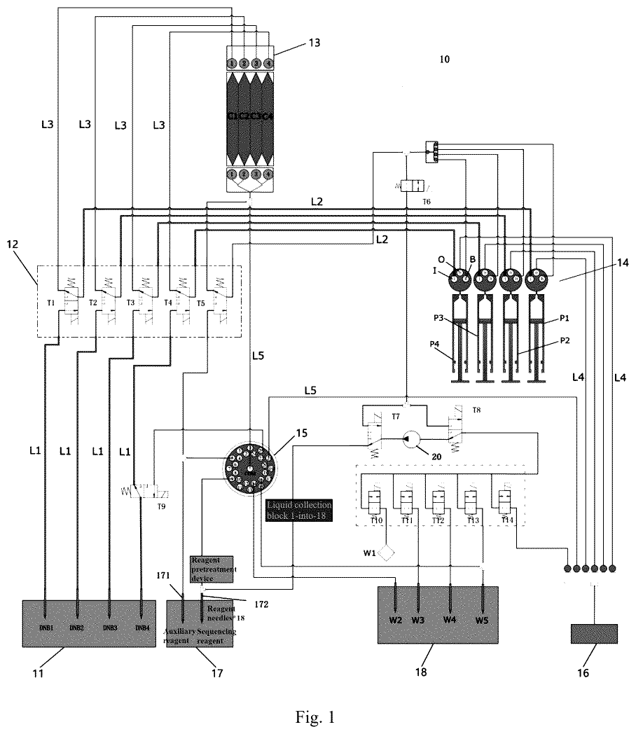

In some embodiments, as shown in FIG. 1, the biological sample area 11 is provided with first to fourth sample storage bins for accommodating the first to fourth biological samples respectively, the first directional control valve assembly 12 includes four first directional control valves T1-T4, the sequencing slide 13 is provided with four first to fourth sample channels C1-C4 independent from each other, and the suction device 14 includes first to fourth suction members P1-P4. The first ports of the four first directional control valves T1-T4 are in communication with the first to fourth sample storage bins through four first pipelines L1 respectively, the second ports of the four first directional control valves T1-T4 are in communication with the I portions (Inlet) of the first to fourth suction members P1-P4 through four second pipelines L2 respectively, and the third ports of the four first directional control valves T1-T4 are in communication with the respective inlet ends of the first to fourth sample channels C1-C4 through four third pipelines L3 respectively. A rotary valve 15 is provided between the outlet ends of the first to fourth sample channels C1-C4 and the waste liquid area 16 for controlling the on-off between the outlet ends of the first to fourth sample channels C1-C4 and the waste liquid area 16. Alternatively, the first directional control valve assembly 12 includes four three-way solenoid valves and a base manifold. Alternatively, the suction device 14 includes four suction pumps, each of which includes a syringe and a valve head, wherein the syringe selectively communicates with one of multiple interfaces of the valve head.

In some embodiments, different biological samples are placed in the first to fourth sample storage bins respectively.

In some embodiments, as shown in FIG. 1, the four first directional control valves T1-T4 are powered on, that is, the first ports of the four first directional control valves T1-T4 are in communication with the second ports respectively, and the second ports are disconnected from the third ports. The I ports of the first to fourth suction members P1-P4 provide negative pressure to suction the first to fourth samples through the first pipeline L1, the four first directional control valves T1-T4 and the second pipeline L2 from the first to fourth sample storage bins respectively, and finally temporarily store biological samples (for example, DNB1-DNB4) in four second pipelines L2 respectively.

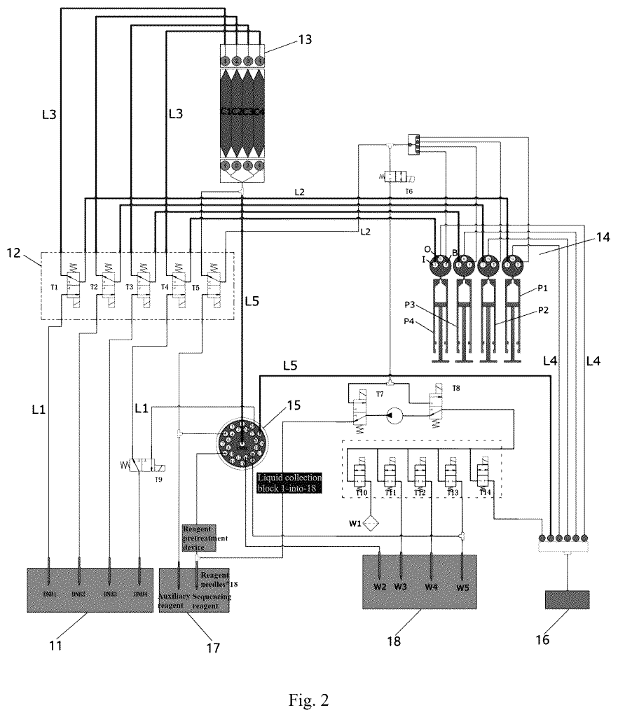

In some embodiments, as shown in FIG. 2, the four first directional control valves T1-T4 are powered off, the second ports of the four first directional control valves T1-T4 communicate with the third port respectively, and the first port is disconnected from the second port. The first to fourth suction members P1-P4 provide positive pressure so that each biological sample temporarily stored in the second pipeline L2 is pushed through the four first directional control valves T1-T4, the third pipeline L3 and the first to fourth sample channels C1-C4, and redundant biological samples are discharged from the outlet ends of the first to fourth sample channels C1-C4 and enter the waste liquid area 16 through the rotary valve 15.

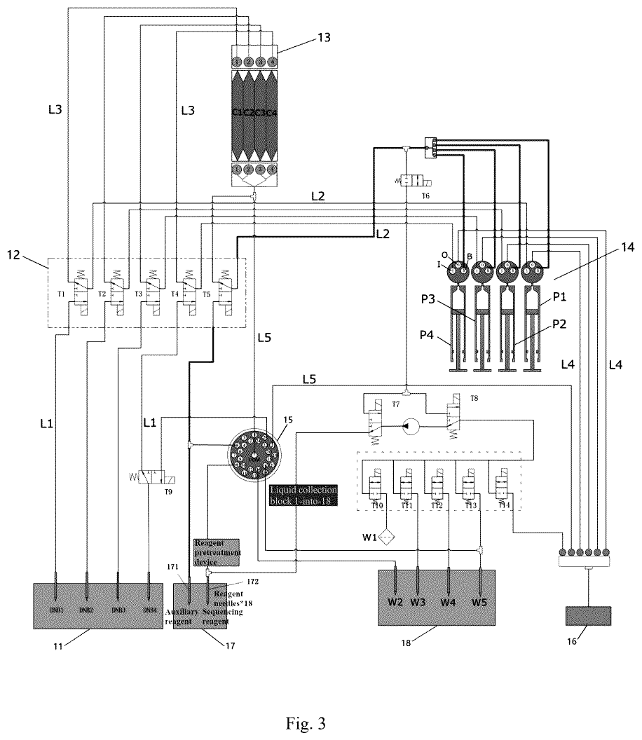

In some embodiments, as shown in FIG. 3, the flow path system for gene sequencing according to the present disclosure includes a sequencing reagent area 17 for storing a sequencing reagent, and the first directional control valve assembly 12 includes a second directional control valve T5. The first port of the second directional control valve T5 communicates with the first sequencing reagent needle 171 of the sequencing reagent area 17, and the first sequencing reagent needle 171 communicates with the sequencing reagent such as an auxiliary reagent. The second port of the second directional control valve T5 communicates with the B ports of the first to fourth suction members P1 to P4, and the third port of the second directional control valve T5 communicates with the outlet ends of the first to fourth sample channels C1 to C4 and the rotary valve 15 through tees respectively. The sequencing reagent area 17 may be partitioned into several reagent storage bins independent of each other as required, and the partition contour line is not shown in the accompanying drawings.

In some embodiments, as shown in FIG. 3, the four first directional control valves T1-T4 are powered off, and the second directional control valve T5 is powered on. The first to fourth suction members P1-P4 provide negative pressure so that the sequencing reagent enters the first to fourth suction members P1-P4 from the sequencing reagent area 17 through the second directional control valve T5 and the B ports of the first to fourth suction members P1-P4 for temporary storage, so as to expel air in the pipeline. The O ports (Outlet) of the first to fourth suction members P1-P4 communicate with the waste liquid area 16 through four fourth pipelines L4.

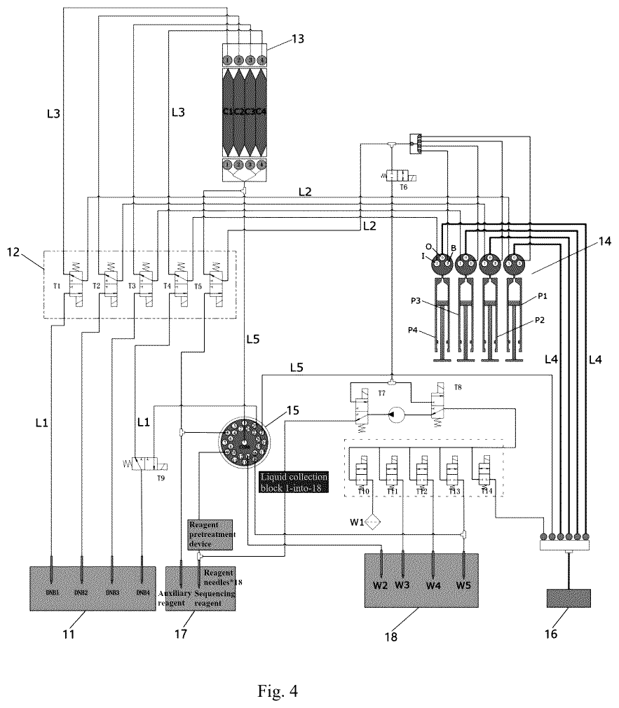

In some embodiments, as shown in FIG. 4, when the first to fourth suction members Pl-P4 are switched to the O port, the first to fourth suction members P1-P4 provide positive pressure to discharge the sequencing reagent temporarily stored in the first to fourth suction members P1-P4 into the waste liquid area 16 through the fourth pipeline L4.

In some embodiments, the four first directional control valves T1-T4 are powered on, and the second directional control valve T5 is powered off. The first to fourth suction members P1-P4 are switched to the I port (Inlet) to provide positive pressure so that the sequencing reagent temporarily stored in the first to fourth suction members P1-P4 is pushed through the second pipeline L2, the four first directional control valves T1-T4 and the first pipeline L1 to reach various samples in the biological sample area 11, so that the first and second pipelines L1 and L2 are filled with the sequencing reagent, and the air in the pipelines is expelled. Refer to FIG. 1 for the flow path.

In some embodiments, the four first directional control valves T1-T4 are powered off, and the second directional control valve T5 is powered on. The rotary valve 15 is switched to the hole position communicating with the waste liquid area 16, and the first to fourth suction members P1-P4 are switched to the I port to provide positive pressure so that the sequencing reagent temporarily stored in the first to fourth suction members P1-P4 is pushed through the second pipeline L2, the four first directional control valves T1-T4, the third pipeline L3, the first to fourth sample channels C1-C4, the fifth pipeline L5 and the rotary valve 15 respectively, and finally enter the waste liquid area 16. In this way, the old sequencing reagents in the first to fourth sample channels C1-C4 are replaced, and the replaced old sequencing reagents and the redundant sequencing reagents are discharged to the waste liquid area 16 from the outlet ends of the first to fourth sample channels C1-C4, so as to prevent that the sequencing reagents in the first to fourth sample channels C1-C4 are contaminated in subsequent test steps. The schematic view of the flow path is similar to FIG. 2.

In some embodiments, only the biological sample is added in the fourth biological storage bin.

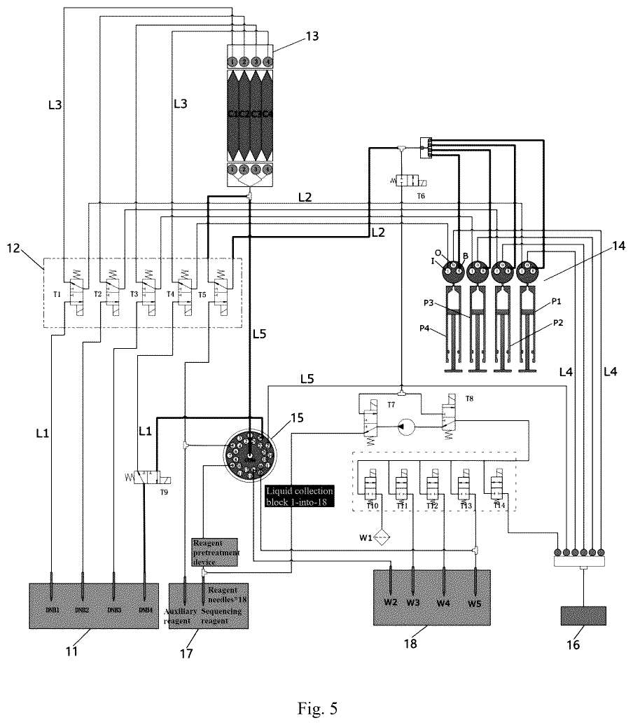

In some embodiments, as shown in FIG. 5, a fifth directional control valve T9 is provided on the first pipeline L1 between the first directional control valve T4 and the fourth sample storage bin, wherein the first port of the fifth directional control valve T9 communicates with the rotary valve 15, the second port of the fifth directional control valve T9 communicates with the fourth sample storage bin, and the third port of the fifth directional control valve T9 communicates with the first port of the first directional control valve T4. In the case where the four first directional control valves T1-T4 and T9 are powered on and the second directional control valve T5 is powered off, the fourth suction member P4 provides negative pressure to suck the fourth biological sample out of the fourth sample storage bin, so as to sequentially pass through the sample needle, the fifth directional control valve T9, the rotary valve 15 and the second directional control valve T5 to reach the B ports of the first to fourth suction members P1-P4. At this time, the fifth pipeline L5 from the rotary valve 15 to the outlet ends of the first to fourth sample channels C1-C4 is filled with the fourth biological sample, thus cleaning the pipeline and filling the biological sample.

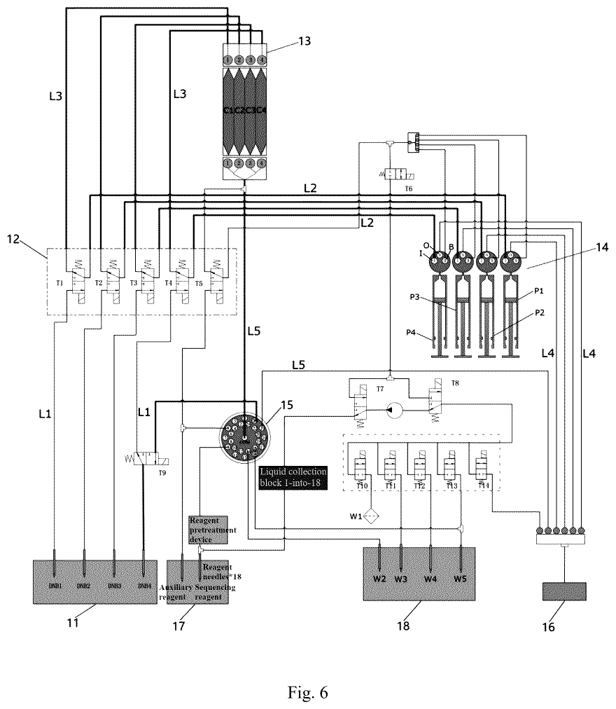

In some embodiments, as shown in FIG. 6, the fifth directional control valve T9 is powered on, the four first directional control valves T1-T4 are powered off, and the second directional control valve T5 is powered on. The first to fourth suction members P1-P4 are switched to the I port to provide negative pressure so that the fourth biological sample in the fifth pipeline L5 is sucked to enter the first to fourth biological sample channels C1-C4 from the outlet end of the sequencing slide 13 respectively, and the redundant fourth biological sample is discharged from the inlet end of the sequencing slide 13, and passes through the third pipeline L3, the four first directional control valves T1-T4 and the second pipeline L2, and through the I ports of the first to fourth suction members P1-P4 to enter the first to fourth suction members P1-P4, thus completing that the fourth biological sample is loaded into the first to fourth sample channels C1-C4. Referring to FIG. 4, the first to fourth suction members P1-P4 are switched to the O port to provide positive pressure so that the biological samples in the first to fourth suction members P1-P4 are pushed and discharged into the waste liquid area 16 through the fourth pipeline L4.

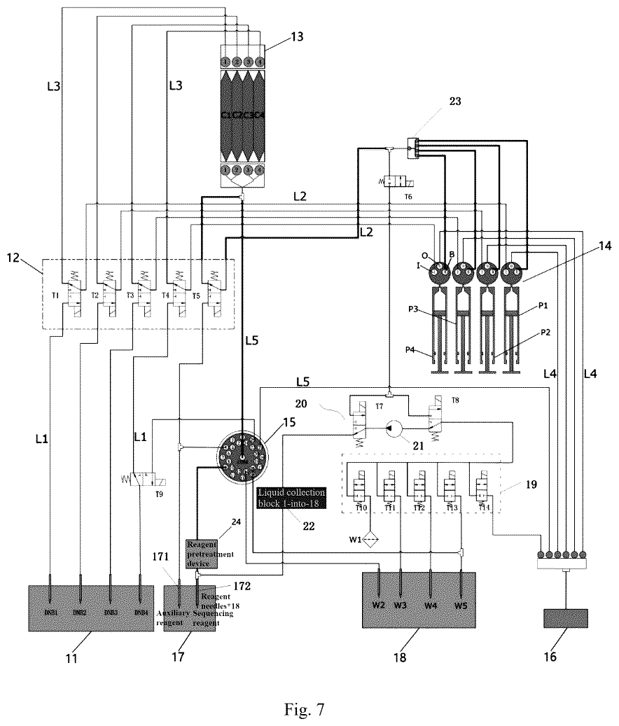

In some embodiments, as shown in FIG. 7, the sequencing reagent area 17 is further provided with a plurality of second sequencing reagent needles 172, which correspond to a plurality of different sequencing reagents respectively. The rotary valve 15 is provided with a plurality of hole positions, which are in one-to-one communication with the plurality of second sequencing reagent needles 172 respectively. When the rotary valve 15 is switched to one of the hole positions, the rotary valve 15 communicates with a second sequencing reagent needle 172 corresponding to this hole position.

In some embodiments, as shown in FIG. 7, a sequencing reagent pretreatment device 24 is provided on a pipeline between the plurality of second sequencing reagent needles 172 and the rotary valve 15, which has at least one of performing filtering, dissolved oxygen removal, degassing and the like on the sequencing reagent extracted from the sequencing reagent area 17.

The sequencing reagent is conveyed by the following two methods: Bypass and Bychip.

The operation steps of Bypass are as follows:
(1) As shown in FIG. 7, the four first directional control valves T1-T4 are powered on, and the second directional control valve T5 is powered off. The rotary valve 15 is switched to communicate with a required sequencing reagent through a second sequencing reagent needle 172, and the first to fourth suction members P1-P4 are switched to the port B to provide negative pressure so that the required sequencing reagent is sucked from the sequencing reagent area 17, and passes through the second sequencing reagent needle 172, the rotary valve 15, the fifth pipeline L5, the second directional control valve T5 and the B ports of the first to fourth suction members P1-P4, so that the required sequencing reagent finally enters the first to fourth suction members P1-P4;
(2) The sequencing reagent may also enter the first to fourth suction members P1-P4 or exceed the second directional control valve T5, as long as the sequencing reagent completely fills the fifth pipeline L5;
(3) The first to fourth suction members P1-P4 are switched to the O port to provide positive pressure so that the sequencing reagent is conveyed from the first to fourth suction members P1-P4 to the waste liquid area 16 through the fourth pipeline L4.

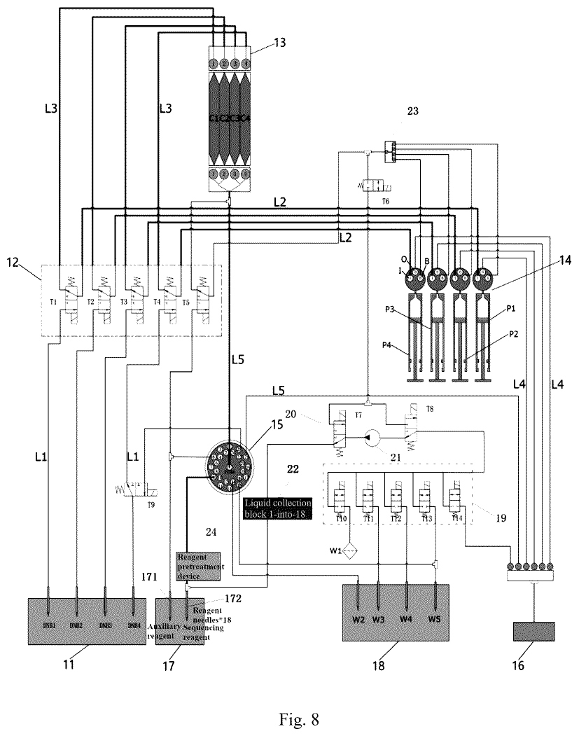

The operation steps of Bychip are as follows:
(1) As shown in FIG. 8, the four first directional control valves T1-T4 are powered off, and the second directional control valve T5 is powered on. The rotary valve 15 is switched to communicate with a required sequencing reagent through a second sequencing reagent needle 172, the first to fourth suction members P1-P4 are switched to the I port, and the first to fourth suction members P1-P4 provide negative pressure to convey the required sequencing reagent through the second sequencing reagent needle 172, the rotary valve 15, the fifth channel L5 and the outlet end of the sequencing slide 13 to enter the biological channels C1-C4 and replace the old sequencing reagents previously therein. The old sequencing reagents and the redundant new sequencing reagents are discharged from the inlet end of the sequencing slide 13, pass through the third pipeline L3, the first to fourth suction members P1-P4, the second pipeline L2 and the I ports of the first to fourth suction members P1-P4, and enter the first to fourth suction members P1-P4.
(2) The first to fourth suction members P1-P4 are switched to the O port to provide positive pressure thrust so that the sequencing reagents inside the same are pushed and discharged into the waste liquid area 16 through the fourth pipeline L4.

One of the following operation steps may be used:
1. Perform all the steps of Bypass, and then perform all the steps of Bychip;
2. Perform steps (1) and (2) of Bypass, and then perform steps (1) and (2) of Bychip; perform steps (1) and (2) of Bypass, then perform step (1) of Bychip, so that the rotary valve 15 is switched to another required sequencing reagent, then perform steps (1) and (2) of Bypass, and next perform steps (1) and (2) of Bychip.

According to the loading flow path of the present disclosure, it is possible to achieve at least one of the following technical effects:

Different biological samples are loaded independently in a plurality of sample channels of the same sequencing slide respectively, which better meets the sequencing requirements of different biological samples and improves the flexibility of gene sequencing;

A plurality of different sequencing reagents were selectively loaded into the selected sample channels respectively to meet the requirements of using different sequencing reagents in different channels in gene sequencing.

### Rinsing Flow Path

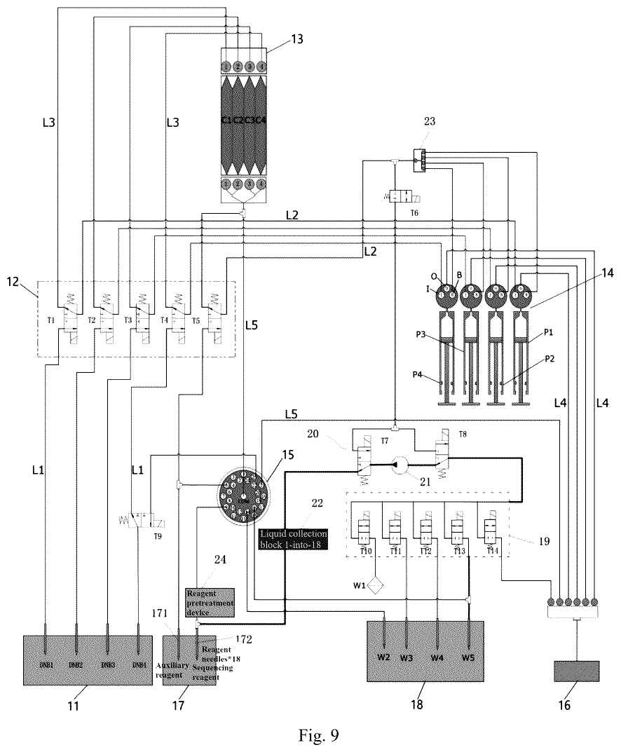

In some embodiments, as shown in FIG. 9, the flow path system for gene sequencing further comprises a rinsing flow path including a rinsing agent area 18 and a second valve group 19. The rinsing agent area 18 stores a plurality of different types of rinsing agents, for example, including a first rinsing agent W1, a second rinsing agent W2, a third rinsing agent W3, a fourth rinsing agent W4 and a fifth rinsing agent W5. The second valve group 19 includes a plurality of inlets and a common outlet, wherein the plurality of inlets communicate with a plurality of different types of rinsing agents respectively. For example, the second valve group 19 includes four two-way valves T10-T13. Each two-way valve has an inlet in communication with a corresponding rinsing agent. The outlet of each two-way valve is connected to form a common outlet, which selectively communicates with the pipeline between the second directional control valve T5 and the B ports of the first to fourth suction members P1-P4 and the outer walls of the plurality of second sequencing reagent needles 172. The rinsing agent area 18 may be partitioned into several rinsing agent storage bins independent of each other as required, and the partition contour line is not shown in the accompanying drawings.

In some embodiments, as shown in FIG. 9, the rinsing flow path includes a second directional control valve assembly, which communicates with the common outlet of the second valve group 19, the pipeline between the second directional control valve T5 and the B ports of the first to fourth suction members P1-P4 and the outer walls of the plurality of second sequencing reagent needles 172 respectively, so that the rinsing agent from the common outlet of the second valve group 19 is communicated with one of the pipeline between the second directional control valve T5 and the B ports of the first to fourth suction members P1-P4 and the outer walls of the plurality of second sequencing reagent needles 172.

In some embodiments, as shown in FIG. 9, the second directional control valve assembly includes a third directional control valve T7, a fourth directional control valve T8 and a pump 21. The common outlet of the second valve group 19 communicates with the second port of the fourth directional control valve T8, the third port of the fourth directional control valve T8 communicates with the inlet of the pump 21, the outlet of the pump 21 communicates with the second port of the third directional control valve T7, the third port of the third directional control valve T7 communicates with the outer walls of the plurality of second sequencing reagent needles 172, the third directional control valve T7 communicates with the first port of the fourth directional control valve T8, and the pipeline between the first port of the third directional control valve T7 and the first port of the fourth directional control valve T8 selectively communicates with the pipeline between the second directional control valve T5 and the B ports of the first to fourth suction members P1-P4 through the cut-off valve T6. Alternatively, the pipeline between the third port of the third directional control valve T7 and the outer walls of the plurality of second sequencing reagent needles 172 is provided with a one-into-multiple liquid dispensing block 22, so as to dispense the rinsing agent from the third port of the third directional control valve T7 to the outer wall of the selected second sequencing reagent needle 172 through the dispensing block 22 so as to rinse the same. Alternatively, the pump 21 may realize speed regulation and forward and backward rotation, and includes, for example, a diaphragm pump and a peristaltic pump.

In some embodiments, as shown in FIG. 9, the second valve group 19 further includes another two-way valve T14, wherein the inlet of the two-way valve T14 communicates with the waste liquid area 16, and the outlet of the two-way valve T14 communicates with the common outlet of the second valve group 19;

The rinsing flow path may perform at least one of the following operation steps:

Step a: one of the two-way valves is selected to be turned on, so that a corresponding rinsing agent is selected, and if the rinsing agent is air, it may be used for emptying a liquid in a pipeline.

Step b: as shown in FIG. 9, the third directional control valve T7 and the fourth directional control valve T8 are powered off, so that the pump 21 rotates forward to transport a selected rinsing agent from the rinsing agent area 18 to the outer wall of the second sequencing reagent needle 172 in the sequencing reagent area 17 through the second valve group 19, the fourth directional control valve T8, the pump 21 and the third directional control valve T7 so as to rinse the outer wall of the second sequencing reagent needle 172.

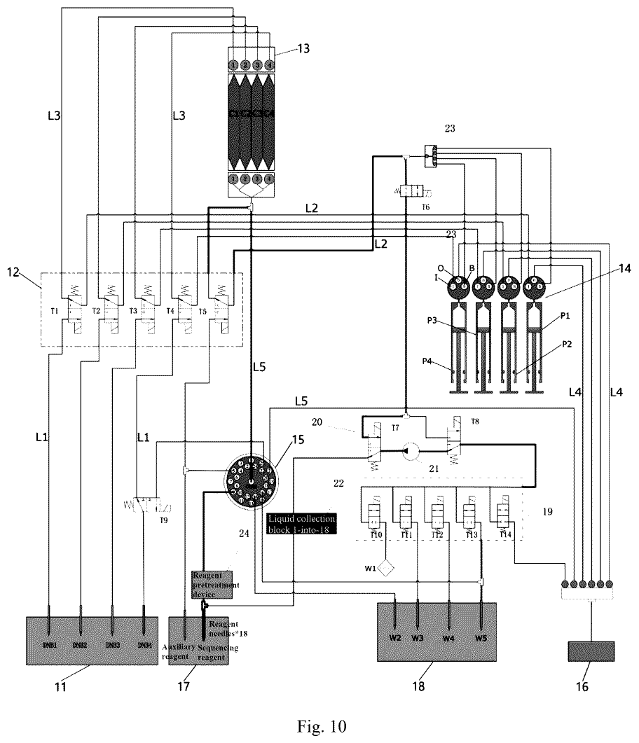

Step c: as shown in FIG. 10, the third directional control valve T7 is powered on and the fourth directional control valve T8 is powered off, and the pump 21 rotates forward so that a required rinsing agent is pumped, and passes through the second valve group 19, the fourth directional control valve T8, the pump 21 and the third directional control valve T7 to reach the cut-off valve T6.

Step d: as shown in FIG. 10, the cut-off valve T6 is turned on, the four first directional control valves T1-T4 are powered on and the second directional control valve T5 is powered off, so that a selected rinsing agent sequentially passes through the cut-off valve T6, the second directional control valve T5, the fifth pipeline L5, the rotary valve 15 and the second sequencing reagent needle 172, and finally enters a sequencing reagent tank in the sequencing reagent area 17.

Step e: the rotary valve 15 is switched to each hole position sequentially, and step d is repeated to complete the rinsing of the inner wall of each second sequencing reagent needle 172 and a communication pipeline thereof.

Step f: step a is performed for multiple times to select different rinsing agents respectively, and step e is performed to rinse the inner wall of each second sequencing reagent needle 172 and a communication pipeline thereof for multiple times with different rinsing agents.

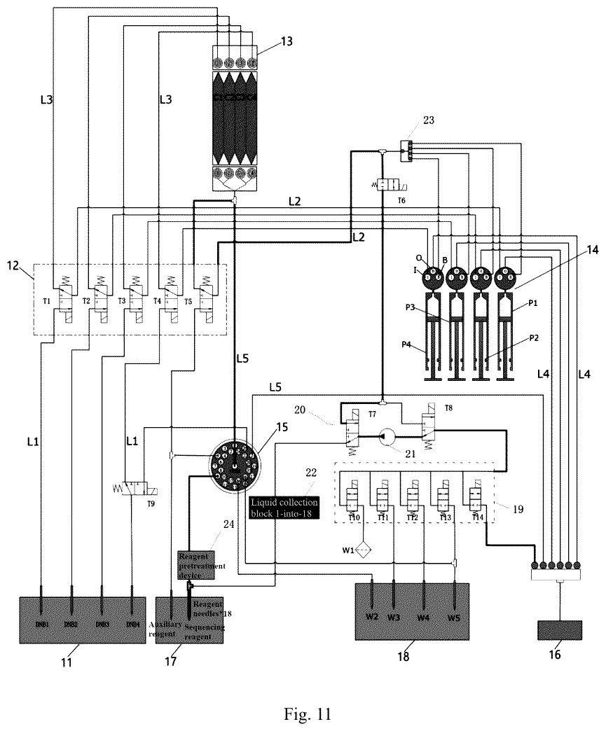

Step g: as shown in FIG. 11, the third directional control valve T7 is powered on and the fourth directional control valve T8 is powered off, the plurality of first directional control valves T1-T4 are powered on and the second directional control valve T5 is powered off so that the pump 21 rotates backward, and only another two-way valve T14 of the second valve group is turned on, and the waste liquid in the sequencing reagent tank of the sequencing reagent area 17 is sucked to the waste liquid area 16.

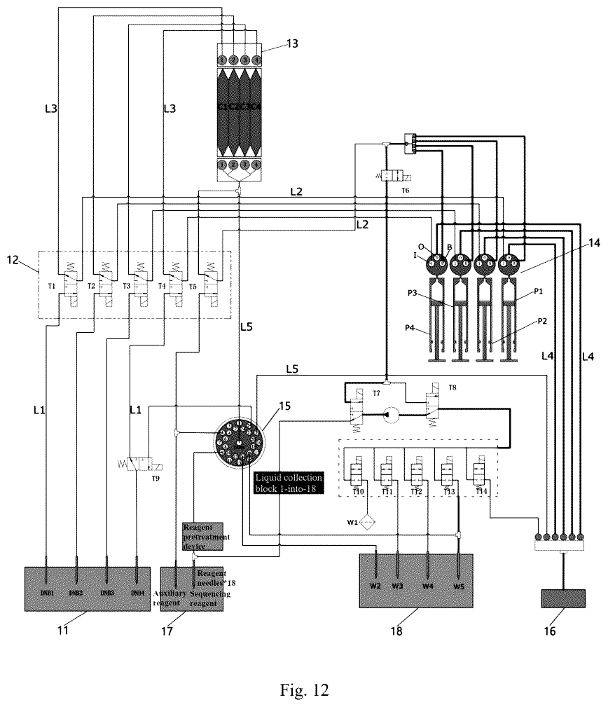

Step h: as shown in FIG. 12, the cut-off valve T6 is turned on, the plurality of first directional control valves T1-T4 are powered on and the second directional control valve T5 is powered off, so that the rotary valve 15 is switched to a blocked hole position, the B and O ports of the first to fourth suction members P1-P4 are simultaneously opened, and the rinsing agent output in step c sequentially passes through the one-into-four liquid dispensing block and the B and O ports of the first to fourth suction members P1-P4, and is finally discharged through the waste liquid area 16, so as to complete the rinsing of related pipelines and valves.

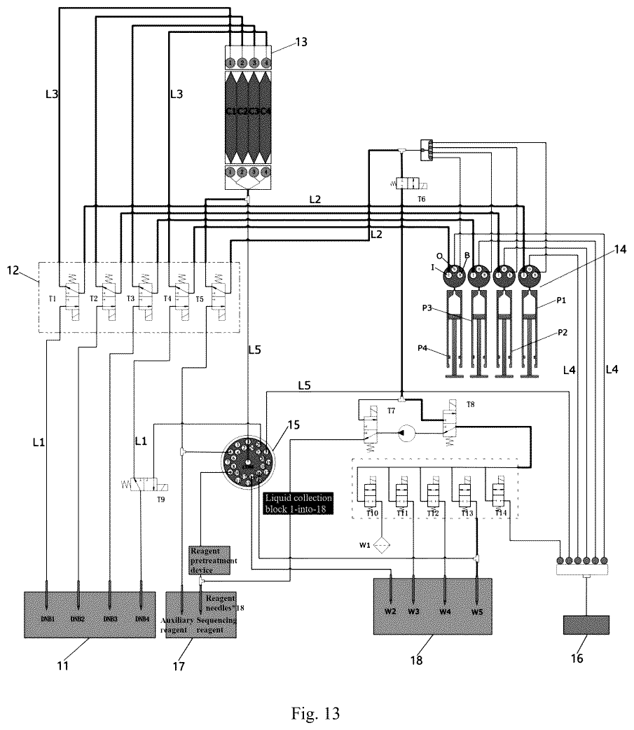

Step i: as shown in FIG. 13, the third directional control valve T7 is powered off and the fourth directional control valve T8 is powered on, and closing the pump 21, so that a selected rinsing agent passes through the outlet of the second valve group 19 and the fourth directional control valve T8 to reach the cut-off valve T6.

Step j: the cut-off valve T6 is turned on, and the first to second directional control valves T1-T5 are powered off, so that the rotary valve 15 is switched to a designated blocked hole position, so that the first to fourth suction members P1-P4 are switched to the I ports to provide negative pressure to suction the rinsing agent which sequentially passes through the cut-off valve T6, the second directional control valve T5, four biological channels C1-C4 of the test slide 13, four directional control valves T1-T4 and the I ports of the first to fourth suction members P1-P4, and finally enters the interior of the first to fourth suction members P1-P4, so as to complete the rinsing of related pipelines and valves.

Step k: the first to four suction members P1-P4 are switched to the O port, and positive pressure is provided to discharge the rinsing agent therein into the waste liquid area 16 through a fourth pipeline L4.

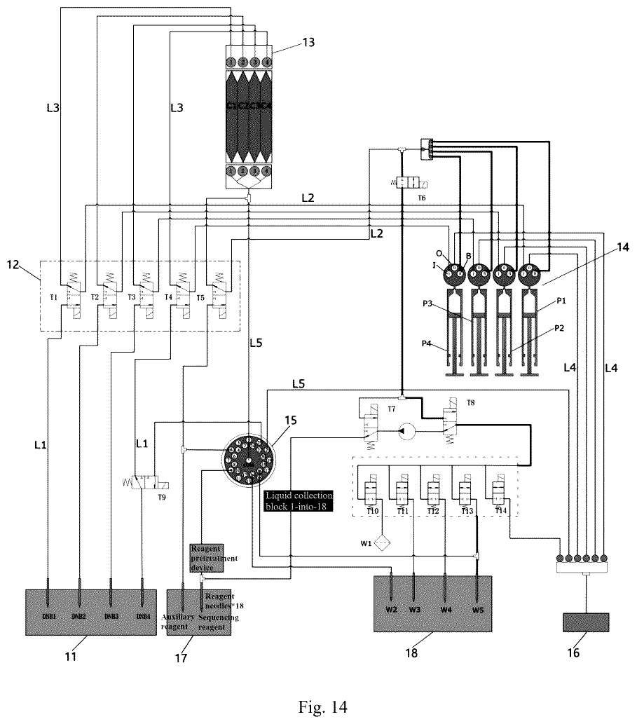

Step l: as shown in FIG. 14, the cut-off valve T6 is turned on, the four first directional control valves T1-T4 are powered on and the second directional control valve T5 is powered off, so that the rotary valve 15 is switched to a designated blocked hole position, and the first to four suction members P1-P4 are switched to the B ports, so as to provide negative pressure to suction the rinsing agent which sequentially passes through the cut-off valve T6, the one-into-four liquid dispensing block 23 and the B ports of the first to four suction members P1-P4, and finally enters the interior of the first to four suction members P1-P4 for temporary storage.

Step m: the four first directional control valves T1-T4 are powered on and the second directional control valve T5 is powered off, so that the first to four suction members P1-P4 are switched to the I port to provide positive pressure to push the rinsing agent, so that the rinsing agent temporarily stored in the first to four suction members P1-P4 in steps j and i is pushed out, and discharged into the first to four sample storage bins in the sample storage area 11 sequentially through the I ports of the first to four suction members P1-P4 and the four first directional control valves T1-T4, and finally through the four sample needles, so as to complete the rinsing of related pipelines and valves.

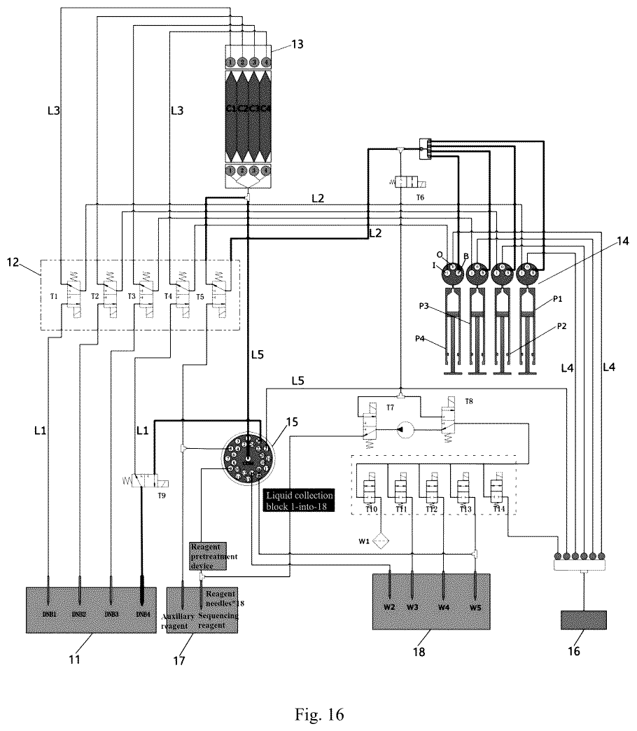

Step n: as shown in FIG. 16, the rotary valve 15 is switched to the hole position in communication with the fifth directional control valve T9, the four first directional control valves T1-T4 are powered on and the second directional control valve T5 is powered off, and the fifth directional control valve T9 is powered on, the first to four suction members P1-P4 are switched to the B port, and positive pressure is provided so that the rinsing agent temporarily stored in the first to four suction members P1-P4 in the step j or i is pushed out, and discharged into a fourth sample storage bin sequentially through the B ports of the first to four suction members P1-P4, the one-into-four liquid dispensing block 23, the second directional control valve T5, the rotary valve 15 and the fifth directional control valve T9, and finally through the sample needle, so as to complete the rinsing of related pipelines and valves.

In the rinsing flow path according to the present disclosure, a plurality of different rinsing agents are used to rinse a flow path member of the flow path system respectively to satisfy different rinsing requirements of each component, thereby improving the rinsing efficiency, saving labor and achieving better rinsing effect.

Finally, it is to be noted that: the above embodiments are only for describing the technical solution of the present disclosure, rather than limiting the same. Although the present disclosure has been described in detail with reference to the foregoing embodiments, those skilled in the art should understand that: it is still possible to modify the technical solutions recited in the foregoing various embodiments, or make equivalent substitutions to some of the technical features. Moreover, these modifications or substitutions do not make the essence of the corresponding technical solutions depart from the spirit and scope of the technical solutions in various embodiments of the present disclosure.

1. A flow path system for gene sequencing, comprising:
a sequencing slide (13) including a plurality of sample channels (C1-C4) independent from each other; and
at least one of the following:
a biological sample loading flow path configured to be capable of loading different biological samples in at least two sample channels respectively, and/or selectively loading one biological sample into some of the plurality of sample channels (C1-C4);
a sequencing reagent loading flow path configured to be capable of selectively loading a plurality of different sequencing reagents into some of the sample channels respectively; and
a rinsing flow path configured to be capable of rinsing a component in the flow path system using a plurality of rinsing agents respectively.

2. The flow path system according to claim 1, further comprising at least one of the following:
a biological sample area (11) provided with a plurality of sample storage bins for storing a plurality of different biological samples;
a sequencing reagent area (17) where the plurality of different sequencing reagents are stored;
a rinsing agent area (18) which stores a plurality of different rinsing agents for rinsing a flow path member of the flow path system; and
a waste liquid area (16) in communication with the biological sample loading flow path, the sequencing reagent loading flow path and the rinsing flow path so as to receive waste liquid.

3. The flow path system according to claim 2, comprising:
a suction device (14) including a plurality of suction members (P1-P4) operating independently of each other for sucking a biological sample;
a first directional control valve assembly (12) including a plurality of first directional control valves (T1-T4) operating independently of each other; and
three ports of each first directional control valve in communication with a corresponding biological sample storage bin, a corresponding suction member and a corresponding sample channel respectively, so as to realize the selective communication of the suction member between the biological sample storage bin and the sample channel.

4. The flow path system according to claim 3, wherein a first port of each first directional control valve communicates with a corresponding biological sample storage bin, a second port of each first directional control valve communicates with an I port of a corresponding suction member, and a third port of the each first directional control valve communicates with an inlet of a corresponding sample channel; and in the case where the first directional control valve is powered on, the first port and the second port of the first directional control valves (T1-T4) are in communication, the second port and the third port of the first directional control valves (T1-T4) are disconnected; and in the case where the first directional control valve is powered off, the second port and the third port of the first directional control valves (T1-T4) are in communication, the first port and the second port of the first directional control valves (T1-T4) are disconnected.

5. The flow path system according to claim 4, further comprising a rotary valve (15) and a fifth directional control valve (T9), wherein a first port of the fifth directional control valve (T9) communicates with the rotary valve (15), a second port of the fifth directional control valve (T9) communicates with a corresponding biological sample storage bin, and a third port of the fifth directional control valve (T9) communicates with a first port of one of the plurality of first directional control valves; the rotary valve (15) communicates with the outlet ends of the plurality of sample channels and the waste liquid area (16); in the case where the fifth directional control valve (T9) is powered on, the first port and the second port of the fifth directional control valve (T9) are in communication, the second port and the third port of the fifth directional control valve (T9) are disconnected; in the case where the fifth directional control valve (T9) is powered off, the second port and the third port of the fifth directional control valve (T9) are in communication, the first port and the second port of the fifth directional control valve (T9) are disconnected.

6. The flow path system according to claim 3, the flow path system is configured to satisfy at least one of the following:
wherein each O port of the plurality of suction members (P1-P4) remains in communication with the waste liquid area (16), so that when the plurality of suction members (P1-P4) are switched to the O port, waste liquid can be discharged into the waste liquid area (16);
wherein the first directional control valve assembly (12) further includes a second directional control valve (T5), a first port of the second directional control valve T5 communicates with a first sequencing reagent needle (171) in the sequencing reagent area (17), a second port of the second directional control valve (T5) communicates with each B port of the plurality of suction members (P1-P4) through a one-into-multiple liquid dispensing block (23), and a third port of the second directional control valve (T5) communicates with the rotary valve (15) and the outlet ends of the plurality of sample channels through tees respectively.

7. (canceled)

8. The flow path system according to claim 5, wherein the sequencing reagent area (17) is provided with a plurality of second sequencing reagent needles (172) which are in communication with the plurality of different sequencing reagents in the sequencing reagent area (17) respectively, and the rotary valve (15) has a plurality of hole positions, each of which is in communication with a corresponding second sequencing reagent needle (172), and the rotary valve (15) is switchable to any of the plurality of hole positions so as to communicate with a corresponding second sequencing reagent needle (172).

9. The flow path system according to claim 8, wherein a sequencing reagent pretreatment device (24) is provided on a pipeline between the second sequencing reagent needle (172) and the rotary valve (15), the sequencing reagent pretreatment device (24) is used for performing at least one of filtering, dissolved oxygen removal and degassing on a sequencing reagent from the sequencing reagent area (17).

10. The flow path system according to claim 8, comprising a second valve group (19) and a second directional control valve assembly (20), wherein the second valve group (19) includes a plurality of inlets and a common outlet, wherein the plurality of inlets are in communication with the plurality of rinsing agents respectively, any of the plurality of inlets may be connected to obtain a required rinsing agent, and the common outlet selectively communicates with one of a pipeline between the second directional control valve (T5) and the B ports of the plurality of suction members and the outer wall of the second sequencing reagent needle (172) of the sequencing reagent area (17) through the second directional control valve assembly (20).

11. The flow path system according to claim 10, wherein the second directional control valve assembly (20) includes a third directional control valve (T7), a fourth directional control valve (T8) and a pump (21), the common outlet of the second valve group (19) communicates with the second port of the fourth directional control valve (T8), the third port of the fourth directional control valve (T8) communicates with the inlet of the pump (21), and the outlet of the pump (21) communicates with the second port of the third directional control valve (T7), and the third port of the third directional control valve (T7) communicates with the outer walls of the plurality of second sequencing reagent needles (172), the third directional control valve (T7) communicates with the first port of the fourth directional control valve (T8), the pipeline between the first port of the third directional control valve (T7) and the first port of the fourth directional control valve (T8) selectively communicates with the pipeline between the second directional control valve (T5) and the B ports of the plurality of suction members through a cut-off valve (T6); and in the case where the third directional control valve (T7) and the fourth directional control valve (T8) are powered on, the first port and the second port of the third directional control valve (T7) are in communication, the second port and the third port of the third directional control valve (T7) are disconnected, the first port and the second port of the fourth directional control valve (T8) are in communication, the second port and the third port of the fourth directional control valve (T8) are disconnected, and in the case where the third directional control valve (T7) and the fourth directional control valve (T8) are powered off, the second port and the third port of the third directional control valve (T7) are in communication, the first port and the second port of the third directional control valve (T7) are disconnected, the second port and the third port of the fourth directional control valve (T8) are in communication, the first port and the second port of the fourth directional control valve (T8) are disconnected.

12. The flow path system according to claim 11, wherein a one-into-multiple liquid dispensing block (22) is provided in the pipeline between the third port of the third directional control valve (T7) and the outer walls of the plurality of second sequencing reagent needles (172) so as to distribute the rinsing agent from the third directional control valve (T7) to the outer walls of the selected second sequencing reagent needle (172).

13. The flow path system according to claim 10, the flow path system is configured to satisfy at least one of the following:
wherein the second valve group (19) includes a plurality of first two-way valves, the inlet of each first two-way valve is in communication with different rinsing agents in the rinsing agent area (18) respectively, and the outlet of each first two-way valve communicates with each other;
wherein the second valve group (19) further includes a second two-way valve (T14), the inlet of the second two-way valve (T14) communicates with the waste liquid area (16), and the outlet of the second two-way valve (T14) communicates with the outlets of the plurality of first two-way valves, so that the waste liquid in the sequencing reagent zone (17) can be discharged to the waste liquid area (16) through the inlet of the second two-way valve (T14) in the case where the pump (21) is reversed.

14. (canceled)

15. A gene sequencer, comprising the flow path system according to claim 1.

16. A method for controlling a flow path system for gene sequencing, wherein the flow path system comprises a biological sample area (11), a first directional control valve assembly (12), a sequencing slide (13), a suction device (14) and a waste liquid area (16), wherein the biological sample area (11) is provided with a plurality of sample storage bins for accommodating a plurality of biological samples respectively, the first directional control valve assembly (12) includes a plurality of first directional control valves (T1-T4), the sequencing slide (13) is provided with a plurality of sample channels (C1-C4), the suction device (14) includes a plurality of suction members (P1-P4), the first ports of the plurality of first directional control valves (T1-T4) are in communication with a plurality of sample storage bins through a plurality of first pipelines (L1) respectively, the second ports of the plurality of first directional control valves (T1-T4) are in communication with I ports of the plurality of suction members (P1-P4) through a plurality of second pipelines (L2) respectively, and the third ports of the plurality of first directional control valves (T1-T4) are in communication with each inlet end of the plurality of sample channels (C1-C4) through a plurality of third pipelines (L3) respectively, and a rotary valve (15) is provided between the outlet ends of the plurality of sample channels (C1-C4) and the waste liquid area (16) for controlling the on-off between the outlet ends of the plurality of sample channels (C1-C4) and the waste liquid area (16); the method comprises:
powering on the plurality of first directional control valves (T1-T4) so that the first ports of the plurality of first directional control valves (T1-T4) are in communication with the second ports respectively;
causing the I ports of the plurality of suction members (P1-P4) to generate negative pressure to suck a plurality of samples from a plurality of sample storage bins respectively to the I ports of the plurality of suction members (P1-P4) through the first pipeline (L1), the plurality of first directional control valves (T1-T4) and the second pipeline (L2), and finally temporarily store the plurality of biological samples in the second pipeline (L2).

17. The method according to claim 16, comprising:
powering off the plurality of first directional control valves (T1-T4), so that the second ports of the plurality of first directional control valves (T1-T4) are in communication with the third ports respectively;
causing the plurality of suction members (P1-P4) to provide positive pressure to push each biological sample temporarily stored in the second pipeline (L2), and pass through the plurality of first directional control valves (T1-T4), the third pipeline (L3) and the plurality of sample channels (C1-C4), so that the respective redundant biological samples are discharged from the outlet ends of the sample channels (C1-C4) and enter the waste liquid area (16) through the rotary valve (15).

18. A method for controlling a flow path system for gene sequencing, wherein the flow path system comprises a biological sample area (11), a first directional control valve assembly (12), a sequencing slide (13), a suction device (14) and a waste liquid area (16), wherein the biological sample area (11) is provided with a plurality of sample storage bins for accommodating a plurality of biological samples respectively, the first directional control valve assembly (12) includes a plurality of first directional control valves (T1-T4), the sequencing slide (13) is provided with a plurality of sample channels (C1-C4), the suction device (14) includes a plurality of suction members (P1-P4), the first ports of the plurality of first directional control valves (T1-T4) are in communication with a plurality of sample storage bins through a plurality of first pipelines (L1) respectively, the second ports of the plurality of first directional control valves (T1-T4) are in communication with I ports of a plurality of suction members (P1-P4) through a plurality of second pipelines (L2) respectively, and the third ports of the plurality of first directional control valves (T1-T4) are in communication with each inlet end of a plurality of sample channels (C1-C4) through a plurality of third pipelines (L3) respectively, and a rotary valve (15) is provided between the outlet ends of the plurality of sample channels (C1-C4) and the waste liquid area (16) for controlling the on-off between the outlet ends of the plurality of sample channels (C1-C4) and the waste liquid area (16); wherein the first directional control valve assembly (12) of the flow path system includes a second directional control valve (T5), the first port of the second directional control valve (T5) communicates with a first sequencing reagent needle (171) in the sequencing reagent area (17), the second port of the second directional control valve (T5) communicates with B ports of the plurality of suction members (P1-P4), and the third port of the second directional control valve (T5) communicates with the outlet ends of the plurality of sample channels (C1-C4) and the rotary valve (15) through tees respectively, wherein in the case where the first directional control valves (T1-T4) and the second directional control valve (T5) are powered on, the first port and the second port of the first directional control valves (T1-T4) are in communication, the second port and the third port of the first directional control valves (T1-T4) are disconnected, the first port and the second port of the second directional control valve (T5) are in communication, the second port and the third port of the second directional control valve (T5) are disconnected, and in the case where first and second directional control valves is powered off, the second port and the third port of the first directional control valves (T1-T4) are in communication, the first port and the second port of the first directional control valves (T1-T4) are disconnected, the second port and the third port of the second directional control valve (T5) are in communication, the first port and the second port of the second directional control valve (T5) are disconnected; the method comprises:
powering off the plurality of first directional control valves (T1-T4) and powering on the second directional control valve (T5); and
causing the plurality of suction members (P1-P4) to provide negative pressure so that the sequencing reagent enters the plurality of suction members (P1-P4) from the sequencing reagent area (17) through the second directional control valve (T5) and the B ports of the plurality of suction members (P1-P4) for temporary storage.

19. The method according to claim 18, the method is configured to satisfy at least one of the following:
wherein in the flow path system, the O ports of the plurality of suction members (P1-P4) are in communication with the waste liquid area (16) through four fourth pipelines (L4) respectively; the method comprises that:
when the plurality of suction members (P1-P4) are switched to the O port, the plurality of suction members (P1-P4) provide positive pressure thrust to discharge a sequencing reagent temporarily stored in the plurality of suction members (P1-P4) into the waste liquid area (16) through the fourth pipeline (L4);
powering on the plurality of first directional control valves (T1-T4) and powering off the second directional control valve (T5); switching the plurality of suction members (P1-P4) to the I port, and providing positive pressure to push a sequencing reagent temporarily stored in the plurality of suction members (P1-P4) and pass through the second pipeline (L2), the plurality of first directional control valves (T1-T4) and the first pipeline L1 to reach each sample needle in the biological sample area (11), so that the first pipelines (L1) and the second pipelines (L2) are filled with a sequencing reagent;
powering off the plurality of first directional control valves (T1-T4) and powering on the second directional control valve (T5); switching the rotary valve (15) to a hole position in communication with the waste liquid area (16), and switching the plurality of suction members (P1-P4) to the I port to provide positive pressure so as to push the sequencing reagent temporarily stored in the plurality of suction members (P1-P4) and pass through the second pipeline (L2), the plurality of first directional control valves (T1-T4), the third pipeline (L3), the plurality of sample channels (C1-C4), the fifth pipeline (L5) and the rotary valve (15) sequentially and finally enter the waste liquid area (16).

20. (canceled)

21. (canceled)

22. The method according to claim 18, a fifth directional control valve (T9) is provided on the first pipeline (L1) between one directional control valve (T4) of the plurality of first directional control valves (T1-T4) and a corresponding sample storage bin, the first port of the fifth directional control valve (T9) communicates with the rotary valve (15), the second port of the fifth directional control valve (T9) communicates with the fourth sample storage bin, the third port of the fifth directional control valve (T9) communicates with the first port of one of the plurality of first directional control valves T4, wherein in the case where the fifth directional control valve (T9) is powered on, the first port and the second port of the fifth directional control valve (T9) are in communication, the second port and the third port of the fifth directional control valve (T9) are disconnected, and in the case where fifth directional control valve is powered off, the second port and the third port of the fifth directional control valve (T9) are in communication, the first port and the second port of the fifth directional control valve (T9) are disconnected,
the method comprises the following steps:
powering on the plurality of first directional control valves (T1-T4) and the fifth directional control valve (T9) and powering off the second directional control valve (T5);
causing one suction member (P4) of the plurality of suction members (P1-P4) to provide negative pressure to suck a biological sample out of a sample storage bin, and sequentially pass through a sample needle, the fifth directional control valve (T9), the rotary valve (15) and the second directional control valve (T5) to reach the B ports of the plurality of suction members (P1-P4), so that a fifth pipeline (L5) from the rotary valve (15) to the outlet ends of the plurality of sample channels (C1-C4) is filled with the biological sample at this time.

23. The method according to claim 22, comprising the following steps:
powering on the fifth directional control valve (T9), powering off the plurality of first directional control valves (T1-T4), and powering on the second directional control valve (T5);
switching the plurality of suction members (P1-P4) to the I port, and providing negative pressure to suck the biological sample in the fifth pipeline (L5) to enter the plurality of biological sample channels (C1-C4) from the outlet end of the sequencing slide (13) respectively, and discharge the redundant biological samples from the inlet end of the sequencing slide (13) and enter the plurality of suction members (P1-P4) through the third pipeline (L3), the plurality of first directional control valves (T1-T4) and the second pipeline (L2) and through the I ports of the plurality of suction members (P1-P4).

24. The method according to claim 22, the sequencing reagent area (17) is provided with a plurality of second sequencing reagent needles (172) corresponding to a plurality of different sequencing reagents respectively; the rotary valve (15) is provided with a plurality of hole positions communicating with the plurality of second sequencing reagent needles (172) respectively; when one of the hole positions of the rotary valve (15) is switched, the rotary valve (15) communicates with one of the second sequencing reagent needles (172) corresponding to the hole position;
the method comprises at least one of Bypass operation and Byship operation:
Bypass operation, which includes the following steps:
powering on the plurality of first directional control valves (T1-T4) and powering off the second directional control valve (T5), switching the rotary valve (15) to communicate with a required sequencing reagent through the second sequencing reagent needle (172), switching the plurality of suction members (P1-P4) to a port B, providing negative pressure to suck the required sequencing reagent from the sequencing reagent area (17), and passing through the second sequencing reagent needle (172), the rotary valve (15), the fifth pipeline L5, the second directional control valve T5 and B ports of the plurality of suction members (P1-P4) so that the sequencing reagent finally enters the plurality of suction members (P1-P4);
causing the sequencing reagent to enter the plurality of suction members (P1-P4), or as long as the sequencing reagent passes through the second directional control valve (T5);
switching the plurality of suction members (P1-P4) to an O port, and providing positive pressure to convey the sequencing reagent from the plurality of suction members (P1-P4) to the waste liquid area (16) through a fourth pipeline (L4); and
Bychip operation, which includes the following steps:
powering off the plurality of first directional control valves (T1-T4), powering on the second directional control valve (T5), switching the rotary valve (15) to communicate with a required sequencing reagent through the second sequencing reagent needle (172), switching the plurality of suction members (P1-P4) to an I port, and causing the plurality of suction members (P1-P4) to provide negative pressure and allowing a required sequencing reagent to pass through the second sequencing reagent needle (171), the rotary valve (15), the fifth channel (L5) and the outlet end of the sequencing slide (13) to enter a biological channel (C1-C4), so as to replace a previous old sequencing reagent therein, so that the old sequencing reagent and redundant new sequencing reagents are discharged from the inlet end of the sequencing slide (13), and pass through the third pipeline (L3), the plurality of suction members (P1-P4), the second pipeline (L2) and I ports of the plurality of suction members (P1-P4) to enter the plurality of suction members (P1-P4);
switching a plurality of suction members (P1-P4) to an O port, and providing positive pressure thrust to push out a sequencing reagent inside the same, and discharge the same into the waste liquid area (16) through a fourth pipeline (L4).

25. (canceled)

26. (canceled)
